(19) United States
(12) Patent Application Publication
(10) Pub. No.: US 2010/0030196 A1
(43) Pub. Date: Feb. 4, 2010

(54) APHERESIS OF A TARGET MOLECULE FROM CEREBROSPINAL FLUID

(75) Inventors: Keith Hildebrand, Houlton, WI (US); Lisa Lynn Shafer, Stillwater, MN (US); Deepak Ramesh Thakker, Blaine, MN (US)

Correspondence Address: CAMPBELL NELSON WHIPPS, LLC, 408 ST. PETER STREET, SUITE 240, ST. PAUL, MN, 55102, US

(73) Assignee: MEDTRONIC, INC., Minneapolis, MN (US)

(21) Appl. No.: 12/511,571

(22) Filed: Jul. 29, 2009

Related U.S. Application Data
(60) Provisional application No. 61/084,279, filed on Jul. 29, 2008.

(51) Int. Cl. A61M 1/16

(52) U.S. Cl. 604/540

(57) ABSTRACT

A system includes (i) a medium having a solid support to which an antibody directed to target molecule is bound, (ii) a reservoir for containing the medium, (iii) a first catheter, and (iv) a second catheter. The first catheter has a proximal end portion, an opening for removal of cerebrospinal fluid from a subject, and a lumen extending from the proximal end portion to the opening. The first catheter is operably couplable to the reservoir such that fluid is capable of flowing from the lumen to the reservoir. The second catheter has a proximal end portion, a delivery region for returning cerebrospinal fluid to the subject, and a lumen extending from the proximal end portion to the delivery region. The second catheter is operably couplable to the reservoir such that fluid is capable of flowing from the reservoir to the lumen.

### FIELD

This disclosure relates to medical devices and methods for removing a target molecule from a body fluid of subject, such as removal of amyloid beta from cerebrospinal fluid.

### BACKGROUND

A variety of disease states are thought to be associated with increased levels of a molecule in a subject. For example, amyloid beta or fibrils or plaques containing amyloid beta are associated with a variety of central nervous system diseases, such as Alzheimer's disease, Lewy body dementia and Down's syndrome. Recent therapeutic strategies designed to decrease plaque burden have focused on immunological approaches, including active and passive immunization targeting amyloid beta. One mechanism that is believed to be involved with these therapeutic strategies is removal of the soluble forms of amyloid beta (monomer and oligomer) from the CNS compartment, which in turn triggers the dissolution of unstable plaques due to a shift in the chemical equilibrium. However, such treatments involve administration of therapeutic substances into the patient and may be associated with risks of producing unintended immunologic and/or inflammatory conditions. To minimize such unintended effects, use of humanized antibodies have been proposed. However, production of such humanized antibodies tends to be costly.

### BRIEF SUMMARY

The present disclosure describes devices, systems and methods that may be employed to accomplish removal of a target molecule, such as soluble amyloid beta components, from a body fluid, such as cerebrospinal fluid (CSF), of a subject and then returning the remaining components of the fluid to the fluid compartment of the subject.

In an embodiment, a system for removing a target molecule, such as amyloid beta, from cerebrospinal fluid is provided. The system includes a medium having a solid support to which an antibody directed to the target molecule is bound. The system also includes a reservoir for containing the medium, a first catheter and a second catheter. The first catheter has a proximal end portion, an opening, and a lumen extending from the proximal end portion to the opening. The first catheter is operably couplable to the reservoir such that fluid is capable of flowing from the lumen to the reservoir. The opening of the first catheter is configured to be placed in the cerebrospinal fluid of a subject. The second catheter has a proximal end portion, a delivery region and a lumen extending from the proximal end portion to the delivery region. The second catheter is operably couplable to the reservoir such that fluid is capable of flowing from the reservoir to the lumen. The delivery region of the second catheter is configured to be placed in the cerebrospinal fluid of the subject. The system may optionally include a pump operably couplable to the first catheter, the second catheter, and the reservoir. The pump, if included, may be configured to cause cerebrospinal fluid to flow from the opening of the first catheter through the reservoir and to the delivery region of the second catheter.

In an embodiment, a method for removing a target molecule from cerebrospinal fluid of a subject is provided. The method includes withdrawing fluid from a first cerebrospinal fluid compartment of the subject and passing the withdrawn fluid through a reservoir of a medical device to remove the target molecule from the withdrawn fluid. The reservoir contains a medium having a solid support to which an antibody directed to the target molecule is bound, and the media is capable of removing the target molecule from the cerebrospinal fluid. The method further includes returning the withdrawn fluid with removed target molecule to a second cerebrospinal fluid compartment of the subject. The first and second cerebrospinal fluid compartments may be the same or different.

In an embodiment, an implantable medical device is provided. The device includes a housing, a reservoir disposed in the housing, and a medium disposed in the reservoir. The medium includes a solid surface to which antibodies directed to a target molecule are bound. The device further includes a fluid outlet operably coupled to the reservoir and a fluid inlet operably coupled to the reservoir. The device also includes a pump disposed in the housing. The pump is operably coupled to the fluid inlet, the fluid outlet, and the reservoir and is configured to cause fluid to flow from the inlet through the reservoir and through the outlet.

Various embodiments of the present invention provide several advantages over known methods and apparatuses for treating neurological disorders. By removing target molecules, such as soluble amyloid beta components, from cerebrospinal fluid (CSF) of a subject and then returning the CSF with removed target molecules back to the patient, delivery of exogenous therapeutic agents can be avoided. By avoiding administration of therapeutic substances, such as antibodies to the subject, side effects including unintended immunologic or inflammatory conditions may be mitigated. These and other advantages will be evident to one of skill in the art upon reading the disclosure herein.

### BRIEF DESCRIPTION OF THE DRAWINGS

FIG. 1 is a schematic block diagram of a system interacting with a cerebrospinal fluid (CSF) compartment of a subject for removing amyloid beta from the CSF.

FIG. 2 is a schematic block diagram of a system interacting with a CSF compartment of a subject, showing details of an embodiment of media for removing amyloid beta from the CSF.

FIG. 3A is a schematic diagram of side view of a representative device and catheter with dashed lines revealing selected components within the device and catheter.

FIG. 3B is a schematic diagram of a longitudinal cross section of the catheter depicted in FIG. 3A.

FIG. 4 is a schematic diagram of a cross section of a brain and spinal cord showing CSF flow.

FIGS. 5-6 are diagrams of a schematic views showing an implanted device and associated catheter in the environment of a subject.

FIG. 7 is a diagram of a schematic view showing an injection port in the environment of a patient.

FIGS. 8-15 are schematic block diagrams of selected components of representative systems.

FIGS. 16A-B are schematic diagrams of views of media having a solid support and a component capable of selectively binding amyloid beta.

The drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

Definitions:

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise.

As used in this specification and the appended claims, the term “or” is generally employed in its sense including “and/or” unless the content clearly dictates otherwise.

As used herein, the terms “treat”, “therapy”, and the like mean alleviating, slowing the progression, preventing, attenuating, or curing the treated disease.

As used herein, “disease”, “disorder”, “condition” and the like, as they relate to a subject's health, are used interchangeably and have meanings ascribed to each and all of such terms.

As used herein, “subject” means a mammal to which an agent, such as an antibody, is administered for the purposes of treatment or investigation. Mammals include mice, rats, cats, guinea pigs, hamsters, dogs, monkeys, chimpanzees, and humans.

As used herein, “couplable” means capable of being coupled, directly or indirectly.

As used herein, “apheresis” means a process of removing a specific component from a fluid of a subject and returning the remaining components to the subject. Often apheresis includes returning the remaining components to same general fluid compartment from which the fluid was removed. For example, if blood is subjected to apheresis to remove a specific component, the remaining components may be returned to blood. If cerebrospinal fluid (CSF) is subjected to apheresis to remove a specific component, the remaining components may be returned to CSF.

The systems and devices described herein may be used for treatment of any disease state for which apheresis of one or more molecules from a body fluid of a subject may be desirable or for investigation of the effects of removal of a molecule (as used herein, “removal” includes a reduction in concentration) from a body fluid of a subject. However for the purposes of brevity and clarity, much of the discussion presented herein is primarily directed to embodiments associated with the treatment or investigation of diseases for which removal of a target molecule from cerebrospinal fluid may be desirable.

Apheresis of a Target Molecule from Cerebrospinal Fluid

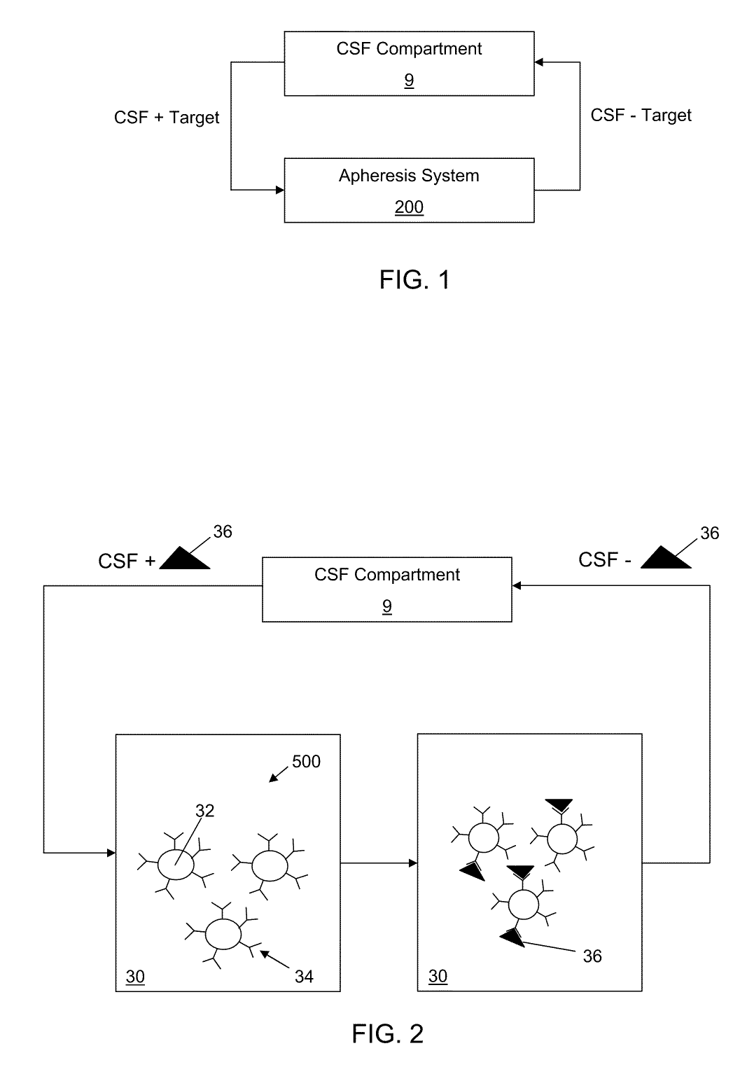

Referring to FIG. 1, an overview of an embodiment of a system is provided. Cerebrospinal fluid (CSF) is removed from a CSF compartment 9 of a subject, one or more target molecules, such as amyloid beta (Aβ), is removed from the CSF by an apheresis system 200 or device, and the CSF with removed target molecule is returned to a CSF compartment 9 of the subject. It will be understood that the CSF compartment 9 from which the CSF is removed may be the same or different compartment 9 to which the CSF is returned.

The apheresis system 200 or device may contain one or more components. In various embodiments, some or all of the components are implantable. In such embodiments, the implantable components having electrical parts are preferably contained within one or more hermetically sealed housings. In some embodiments, some or all of the components are external to the subject.

Referring now to FIG. 2, the apheresis system may include a reservoir 30 containing a medium 500 having a solid support 32 with which an antibody 34 or other binding partner that is capable of selectively binding target molecule 36 is associated. CSF containing the target molecule 36 is removed from a CSF compartment 9 of a subject, and the CSF is flowed through the reservoir 30 and contacted with the medium 500. The antibody 34 or other binding partner selectively binds the target molecule 36, removing some or all of the target molecule 36 from the CSF. The CSF with removed target molecule 36 is then returned to a CSF compartment 9, which may the same or different compartment 9 from which the CSF was removed.

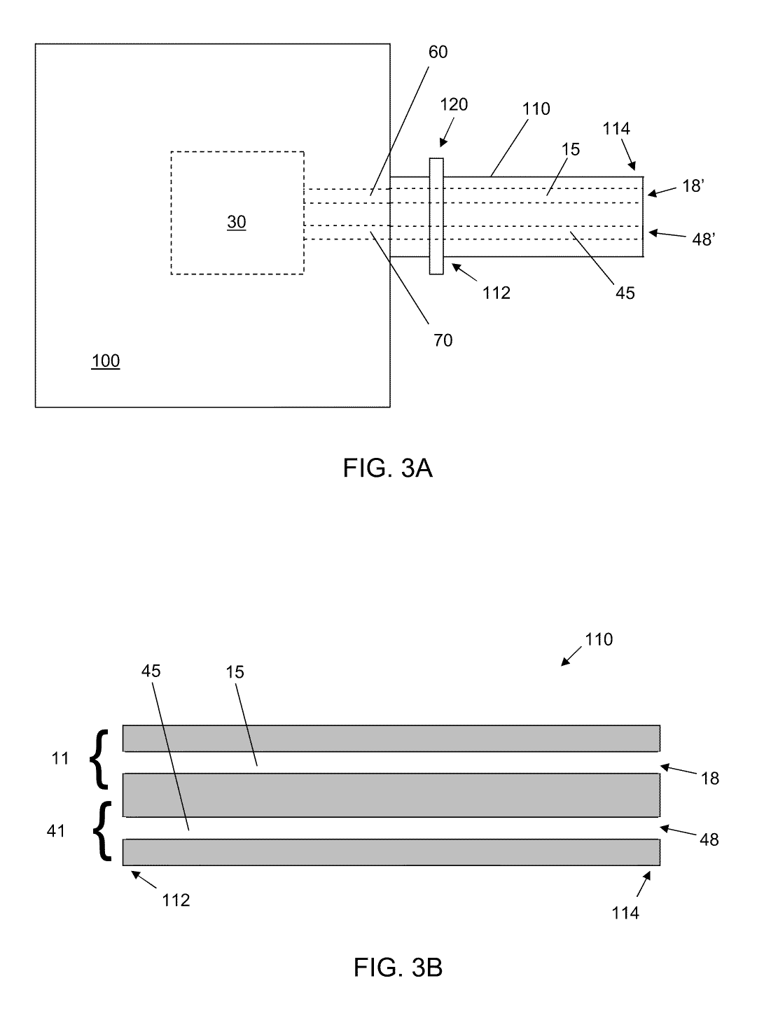

Referring now to FIG. 3A, a schematic diagram of a side view of a representative device 100 having a reservoir 30 operably coupled to a catheter 110 is shown (dashed lines indicate portions of device 100 or catheter 110 within and beneath the exterior surface the device or catheter). The device 100 may include a connector 120 to which proximal end 112 of catheter 110 may be connected. Catheter 110 may be connected to connector 120 via any suitable mechanism, such as clamping, compression fitting, interference fit, or the like. In the depicted embodiment, catheter 110 has a first lumen 15 and a second lumen 45. The first lumen 15 extends through the catheter 110 from proximal end portion 114 to an opening 18 at distal end portion 114. While not show, it will be understood that the opening 18 may be located at any position along the catheter 110 and there may be more than one opening. The first lumen 15 is operably coupled to an inlet 60 of device 100. While not shown, it will be understood that one or more components, such as a valve, a pump, or the like, may be located between the fluid flow path of inlet 60 and reservoir 30. The system is configured such that CSF may flow through opening 18, through first lumen 15, through inlet 60 and into reservoir 30.

Still referring to FIG. 3A, catheter 110, in the depicted embodiment, has a second lumen 45 that extends through the catheter 110 from the proximal end 112 to a delivery region 48 at distal end portion 114. While not shown, it will be understood that the delivery region 48 may be located at any position along the catheter 110 and there may be more than one delivery region. The second lumen 45 is operably coupled to an outlet 70 of device 100. While also not shown, it will be understood that one or more components, such as a valve, a pump, or the like, may be located between the fluid flow path of outlet 70 and reservoir 30. The system is configured such that CSF may flow from reservoir 30 through outlet 70, through second lumen 45 and out through delivery region 48.

CSF may enter reservoir 30, which contains a medium for removing a target molecule (see FIG. 2), via the first lumen 15 of catheter 110 and may exit reservoir 30 and be returned to a subject via delivery region 48 of catheter 110. While moving through reservoir 30, target molecules are removed from the CSF, and CSF with reduced levels of the target molecule is returned to the subject.

FIG. 3B depicts a schematic drawing of a longitudinal cross section of the catheter 110 shown in FIG. 3A. The depicted catheter 110 includes a first catheter 11 and a second catheter 41. In various embodiments, the first and second catheters 11, 41 are integrally formed in a single catheter 110. The first catheter 11 includes a lumen 15 extending through the catheter 11 from the proximal end portion 112 to the opening 18.

The second catheter 41 includes a lumen 45 extending through the catheter 41 from the proximal end portion 112 to the delivery region 48. While a single catheter 110 with two lumens 15, 45 (or two catheters 11, 41) is depicted in FIG. 3, it will be understood that two separate catheters, or a catheter that splits into two separate catheters at some point along its length, may be employed.

Cerebrospinal Fluid Compartment

In accordance with the teachings presented herein, CSF may be removed from or returned to any CSF compartment of a subject. One suitable CSF compartment for removal and return of CSF is the subarachnoid space. Cerebrospinal fluid is produced in the ventricular system of the brain and communicates freely with the subarachnoid space via the foramina of Magendie and Luschka.

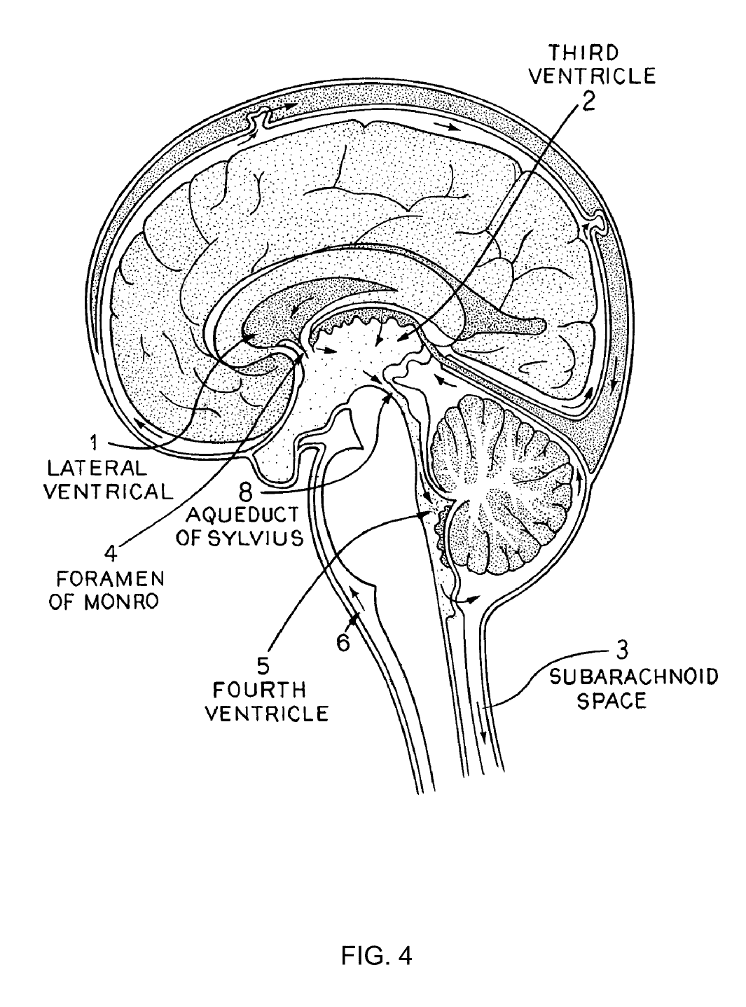

As illustrated in FIG. 4, the central nervous system (brain and spinal cord) is surrounded by cerebrospinal fluid 6 contained within the subarachnoid space 3. In addition, cerebrospinal fluid 6 is also contained in the four ventricles of the brain: two lateral ventricles 1, the third ventricle 2, and the fourth ventricle 5. The lateral ventricles 1 are connected to the third ventricle 2 via the foramen of Monro 4; the third ventricle 2 is connected to the fourth ventricle 5 via the aqueduct of Sylvius 8. The arrows within the subarachnoid space 3 in FIG. 4 indicate cerebrospinal fluid 6 flow.

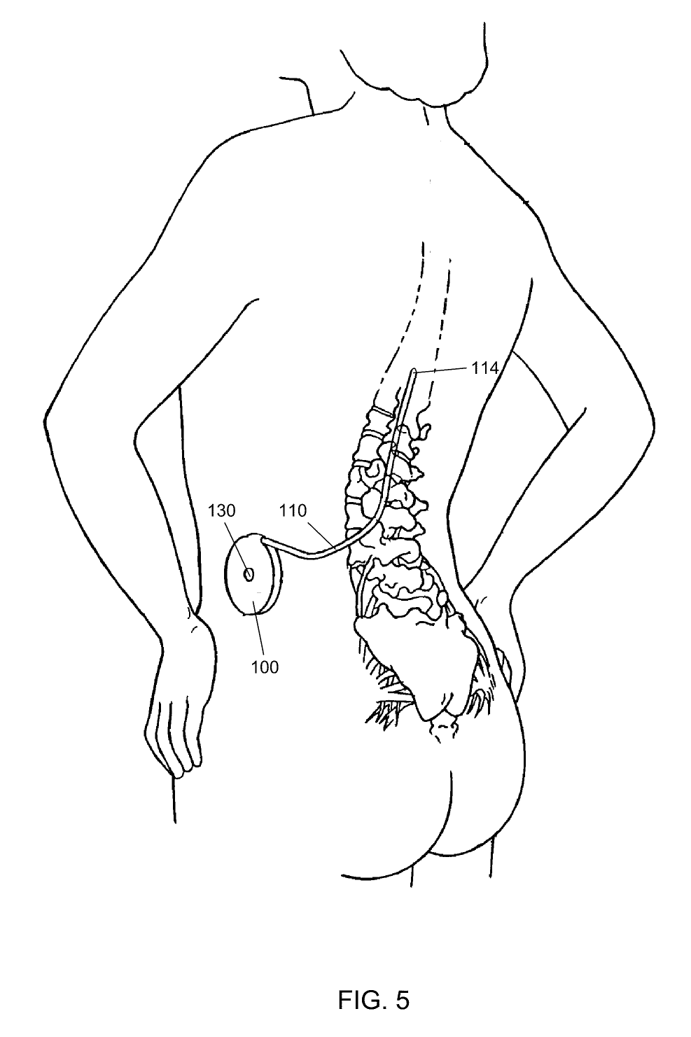

According to various embodiments, CSF is obtained from, or returned to, the spinal canal of a subject. With reference to FIG. 5, a system similar to that depicted in FIG. 3A is shown implanted in a patient. The system includes a device 100 containing a reservoir (not shown in FIG. 5) and catheter 110 operably coupled to the device 100. Distal portion 114 of catheter 110 is shown implanted in the intrathecal space of the patient's spinal canal. One or more openings (not shown in FIG. 5) for receiving CSF and one or more delivery regions (not shown in FIG. 5) for returning CSF are located at or near distal portion 114 of catheter 110. In the embodiment depicted in FIG. 5, device 100 is implanted below the skin of the patient. Preferably the device 100 is implanted in a location where the implantation interferes as little as practicable with activity of the patient. One suitable location for implanting the device 100 is subcutaneously in the lower abdomen. The device 100 may include a port 130 configured to fluidly communicate with reservoir (not shown in FIG. 5). A needle or other suitable device may be inserted into port 130 to inject to withdraw fluids from the reservoir, allowing for replacement or regeneration of the medium for removing a target molecule, as described in more detail below. Preferably, device 100 is implanted subcutaneously in a manner such that port 130 may be percutaneously accessed by a needle.

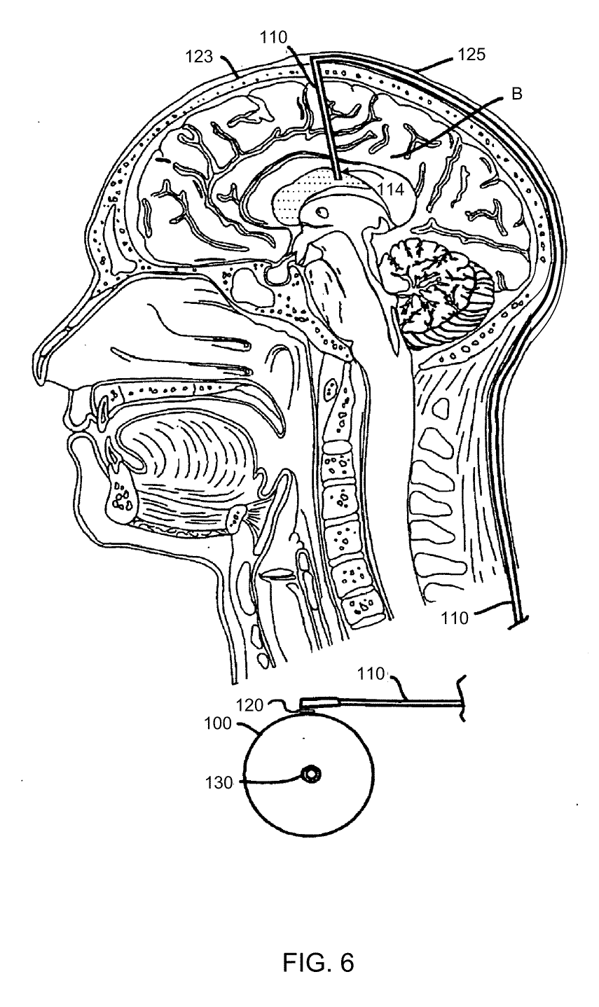

According to various embodiments, CSF may be withdrawn from, or returned to, a ventricle of the brain of a subject. Referring to FIG. 6, a device 100 having a reservoir (not shown in FIG. 6) containing media for removing a target molecule from the subject's CSF may be implanted below the skin of a subject. As with the device depicted in FIG. 5, the device 100 depicted in FIG. 6 may have a port 130 through which the reservoir may be accessed. In the depicted embodiment, the distal end 114 of catheter 110 terminates in a ventricle of the brain. Distal end portion 114 of catheter 110 may be implanted in the ventricle using conventional stereotactic surgical techniques. The distal portion 114 is surgically introduced through a hole in the skull 123 and a mid portion of catheter 110 may be implanted between the skull and the scalp 125 as shown in FIG. 6. Catheter 110 may be joined to implanted device 100, for example, via connector 120.

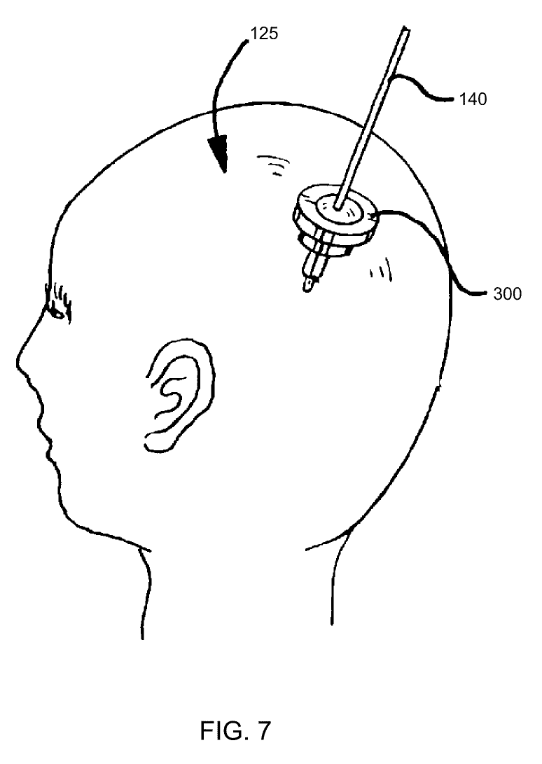

In various embodiments, CSF is removed from a subject, contacted with a medium for removing a target molecule from the CSF where the medium is contained in a reservoir external to the patient, and returned to the subject. The CSF may be removed from, or returned to, the subject via the subject's intrathecal space, intraventricular space, or the like. Referring to FIG. 7, CSF may be withdrawn from, or returned to, a subject's CNS via an injection port 300 implanted subcutaneously in the scalp of a patient 125, e.g. as described in U.S. Pat. No. 5,954,687 or otherwise known in the art. A guide catheter 140 may be used to guide a catheter for removing or returning CSF from the brain of the subject. Of course, a catheter for removing or returning CSF may be directly inserted through port 300 to the target location.

Any other known or developed implantable or external infusion device, port, shunt, or the like may readily be adapted for apheresis of amyloid beta from CSF.

Representative Device Configurations

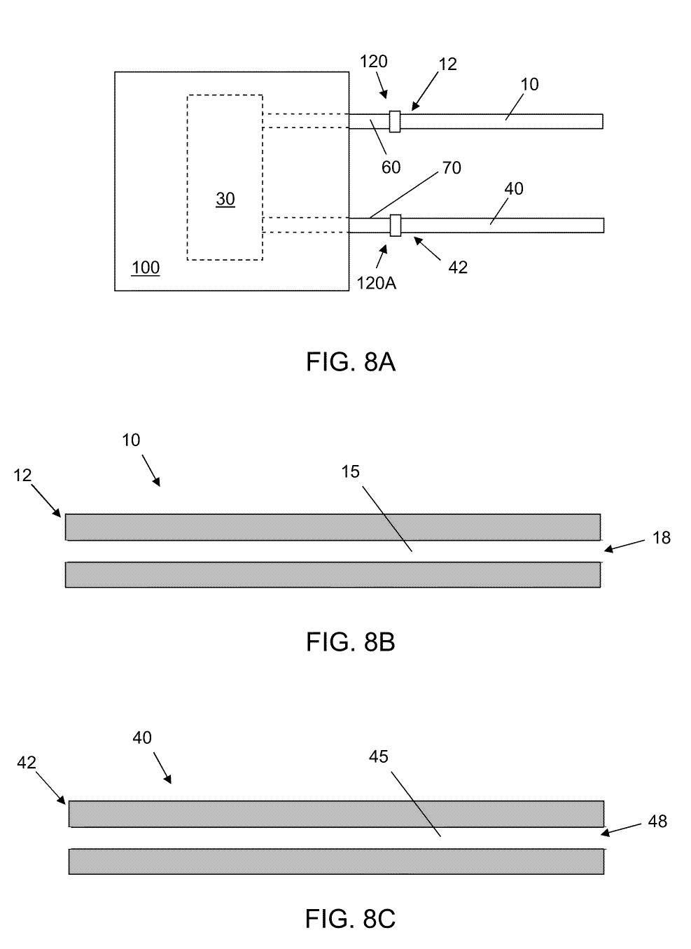

FIGS. 3A-B, 5 and 6, discussed above, and FIGS. 8-15 provide representative examples of device configurations that may be employed for apheresis of target molecule from CSF of a subject. It will be understood that components in addition to those depicted in FIGS. 3A-B, 5, 6 and 8-14 may be employed. However, selected components are shown for sake of clarity and brevity.

Referring to FIG. 8A, a device 100 having a reservoir 30 for containing a medium for removing amyloid beta from CSF is shown. The device 100 is similar to the device depicted in FIG. 3A, except that the device 100 in FIG. 8 is coupled to two catheters, a first catheter 10 or tube and a second catheter 40 or tube. The device 100 contains an inlet 60 and outlet 70 operably coupled to reservoir 30. A first catheter 10, a cross section of which is shown in FIG. 8B, is operably couplable to the device 100, e.g. by securing via connector 120, such that lumen 15 of first catheter 10 can be in fluid communication with inlet 60. A second catheter 40, a cross section of which is shown in FIG. 8C, is operably couplable to the device 100, e.g. by securing via connector 120A, such that lumen 45 of second catheter 40 can be in fluid communication with outlet 70. The system depicted in FIG. 8 is capable of operating such that, when opening 18 of first catheter 10 and delivery region 48 of second catheter 40 are placed in a CSF compartment of a subject, CSF may flow into opening 18, through lumen 15 of first catheter 10, through inlet 60, through reservoir 30, through outlet 70, through lumen 45 of second catheter 40, and out of delivery region 48. While CSF flows through reservoir 30 a medium contained within the reservoir can remove a target molecule from the CSF so that CSF with a reduced target molecule concentration may be returned to a CSF compartment of the subject.

While not shown in FIG. 8, it will be understood that the opening 18 may be located at any suitable position along the first catheter 10 and there may be more than one opening. In addition and while not shown, it will be understood that the delivery region 48 may be located at any suitable position along the second catheter 40 and there may be more than one delivery region. It will be further understood that one or more components, such as a valve, a pump, or the like, may be located between the fluid flow path of inlet 60 and reservoir 30 or outlet 70 and reservoir 30.

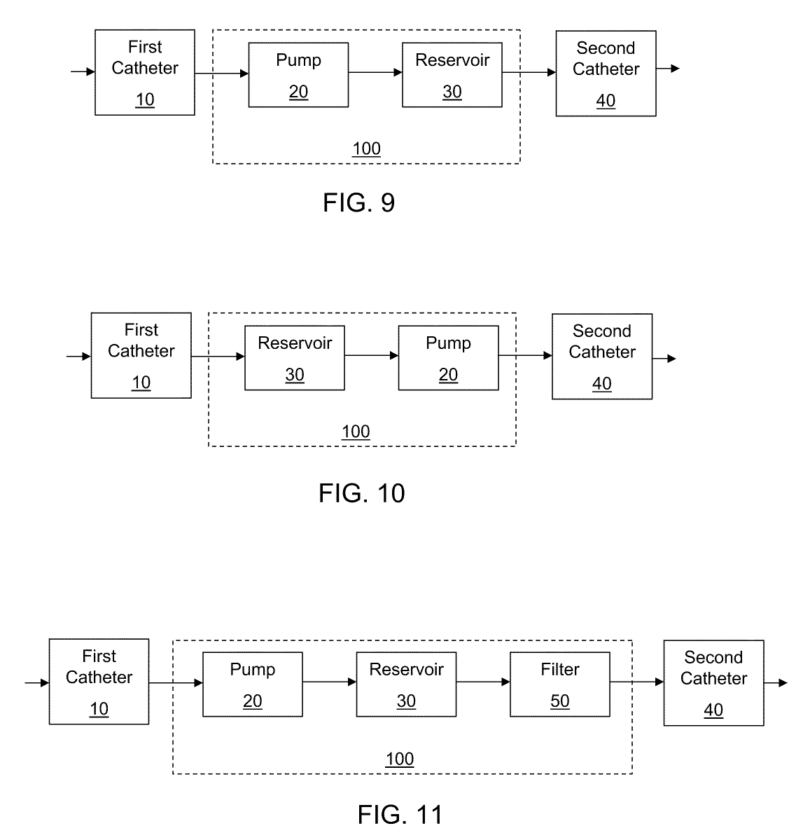

Referring now to FIGS. 9-12, block diagrams of representative systems are shown. The systems include a device 100 having a reservoir 30 and a pump 20 are shown. In some embodiments, apheresis systems do not include a pump. It is believed that the pulsatile nature of CSF flow may be sufficient to force CSF to flow through an apheresis system and back to CSF without use of a pump. In addition, or alternatively, gravity may be used to assist in pumpless devices; e.g. CSF may be removed from a higher CNS level and returned to a lower CNS level. While such pumpless systems and devices may be advantageous in some situations, it may be desirable, in some situations, to employ a pump; e.g., when media for removal of the target molecule restricts flow or to increase the amount of the target molecule removed by increasing the flow through the media. Any suitable pump 20 may be employed. For example, the pump 20 may be a peristaltic pump, an osmotic pump, a piston pump, a diaphragm pump, or the like. The pump 20 may be fixed rate, variable rate, programmable, etc.

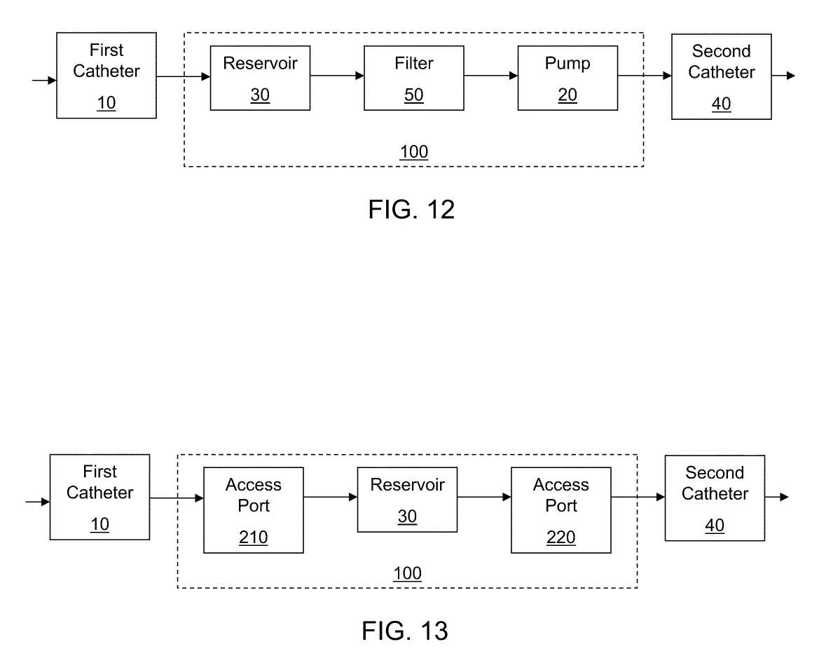

The systems depicted in FIGS. 9-12 further include a first catheter 10 and a second catheter 40, which may be a single catheter having two lumens (see, e.g., FIG. 3) or may be separate catheters (see, e.g., FIG. 8), operably coupled to the reservoir 30. The direction of the arrows in FIGS. 9-12 indicates the desired direction of flow of CSF through the system. As shown in FIG. 9 and FIG. 11, the pump 20 may be located upstream of the reservoir 30, i.e. between the reservoir 30 and the first catheter 40. Alternatively, as shown in FIGS. 10 and 12, the pump 20 may be located downstream of the reservoir 30, i.e. between the reservoir 30 and the second catheter.

The device may further include a filter 50. The filter 50 is configured to prevent selected components of media, such as a solid support bead, contained in the reservoir 30 from entering the subjects CSF via second catheter 40. In various embodiments, as described above, the media contains solid support material that may flow with CSF. In such embodiments, a filter 50 may be desirable. In some embodiments, the media contains a solid support that is not likely to flow or will not flow with CSF as the CSF passes through the reservoir 30. In such embodiments, it may be desirable to omit a filter 50 from the device. Preferably, the filter 50 is positioned such that it prevents selected components of the media from leaving the reservoir 30. In various embodiments, filter 50 is positioned within the reservoir 30 or immediately downstream of the reservoir 30. Filter 50 may be made of any suitable material, such as poly(tetrafluoroethane) (PTFE), nylon, cellulose, mixed cellulose ester, or polyvinylidene difluoride (PVDF). Preferably, the pore size of filter 50 is small enough to retain the solid support, such as beads. For example, the pore size may be about 20 to about 50 microns less in diametric dimension than the diametric dimension of the solid support. In various embodiments, it may be desirable for the filter to exhibit a low affinity for binding to protein. One suitable low protein binding material is PVDF. In some embodiments, a filter 50 may serve as a solid support for an antibody for binding amyloid beta.

Referring now to FIG. 13, a block diagram of a system having access ports 210, 220 upstream and downstream of the reservoir 30 is shown. Access ports 210 may be used to sample CSF before it enters the reservoir; e.g. to assess the level of target molecule in the CSF prior to apheresis. Access port 220 may be used to sample CSF after apheresis; e.g. to determine how effectively the target molecule is being removed from the CSF by the device 100. CSF removed via access portion 210, 220 may be used to determine the effects of apheresis on molecules other than the target molecules, to monitor or diagnose a condition of the subject, to determine when the media for removal of the target molecule is saturated, or the like. For example, the concentration of a target molecule or other molecule in fluid sampled from an access port 210, 220 may be quantitatively or semi-quantitatively determined via a suitable assay or device, such as an ELISA assay or microchip based bioassay. The amount of target or other molecule may be used to determine whether apheresis is effectively removing the target molecule or having a desirable or expected effect on another molecule, may be used to determine whether the apheresis media is, or is becoming, saturated, or the like. Apheresis parameters may be altered based on information regarding concentration of the target or other molecule. For example, if an apheresis device includes a variable rate or programmable pump, the rate at which fluid is flows through the apheresis media may be changed to remove more (increase flow) or less (decrease flow) of the target molecule from the CSF of the patient.

In some embodiments (not shown in FIG. 13), a system includes only one access port for sampling CSF. The single access port may be upstream, downstream, or at the reservoir.

While not shown in FIGS. 9-13, it will be understood that the devices 100 may include further components such as an inlet, an outlet, a microprocessor for controlling the pump, a sensor module, a telemetry module, a diagnostics module, a power supply, a reservoir access port, etc.

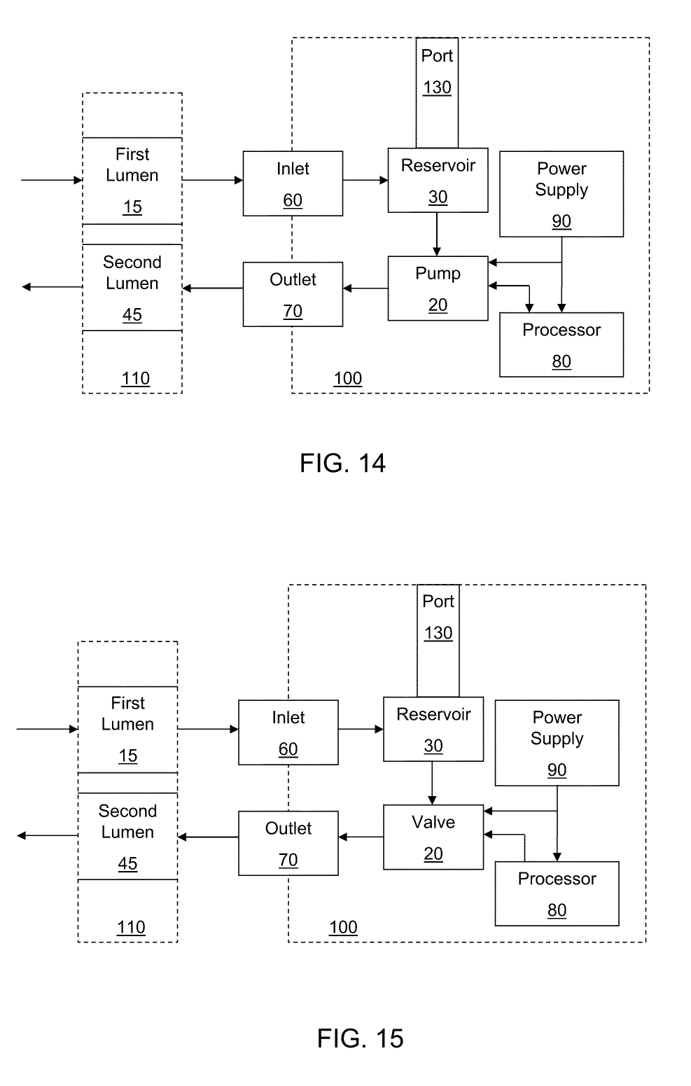

Referring now to FIG. 14, a block diagram of a system is shown. The system includes a catheter 110 through which CSF may flow and a device 100 for removing a target molecule from the CSF. The device 100 includes a reservoir 30 and a pump 20 operably coupled to the reservoir 30 and configured to cause CSF to move through the reservoir 30. The device 100 further includes a processor 80 operably coupled to the pump 20 and configured to control the rate at which the pump 20 causes CSF to move through the reservoir 30. The device also includes a power supply 90 operably coupled to the processor 80. The power supply may also be operably coupled to the pump 20 in embodiments where it is desirable to provide power for one or more aspects of pump operation.

With reference to FIG. 15, a block diagram of an alternative embodiment of a system is shown. The system includes first 10 and second 40 catheters through which CSF may flow operably coupled to a device 100 for removing a target molecule from the CSF. The device 100 includes a valve 220 configured to control the rate of flow of CSF through the device 100. The valve 220 is operably coupled to reservoir 30. While only one valve 220 is depicted, it will be understood that device 100 may include more than one valve. Power supply 90 and processor 80 are operably coupled to valve 220. Processor 80 may be configured to control the rate at which CSF may flow through the valve 220, e.g. by causing the valve 220 to open or close, partially or entirely, as instructed. While not shown, it will be understood that device 100 may include a pump or a sensor for detecting flow. The sensor may be coupled to the processor 80 to allow for closed loop control of valve 20.

The devices 100 shown in FIGS. 14-15 also include a port 130 for accessing the reservoir 30. A needle (not shown) may be introduced into port 130 to introduce or remove fluid from the reservoir 30.

The device and system configurations described herein are representative examples of configurations that may be employed. It will be understood that the various system components shown in FIGS. 3A-B, 5, 6, and 8-15 may be readily interchanged as desired. For example, an access port 210, 220 depicted in FIG. 13 may be readily introduced into a device or system configuration depicted in FIGS. 3A-B, 5, 6, 8-12, 14 or 15. It will be also be understood that other configurations and other components are readily obtainable and are contemplated for use with the teachings described herein.

Target Molecule

The apheresis systems described herein may be used to treat any disease in which removal of a target molecule from a bodily fluid may be beneficial or to investigate the effects of removal of a target molecule from a body fluid of a subject (e.g., in experimental animals). For the purposes of brevity, much of this disclosure is limited to a discussion regarding removal of a target molecule from CSF and diseases for which such removal may be beneficial, such as Alzheimer's Disease (AD), Lewy body dementia, and Down's Syndrome. Any one or more target molecules may be removed from CSF via apheresis as described herein. For the purposes of brevity a few examples of target molecules that may be removed from CSF are discussed below.

In various embodiments, the target molecule to be removed from CSF via apheresis is tau. Tau is a microtubule-associated protein that is found mostly in neurons. One function of tau is to modulate the stability of axonal microtubules. However, hyperphosphorylation or excessive tau activity may result in self-assembly of tangles of paired helical filaments or straight filaments, thought to be involved in AD and other diseases. Accordingly, apheresis of tau may result in a reduction of self-assembly of tangles. To date there are six known isoforms of tau. Any one or more of the six isoforms of tau may be a target molecule for apheresis as described herein. Phosphorylated or unphosphorylated tau may be removed via apheresis.

In various embodiments, one or more cytokines, such as interleukin (IL)-11, IL-18, or tumor necrosis factor-alpha (TNFα), may be a target molecule for CSF apheresis, as intrathecal inflammation has been reported to precede development of AD. Some anecdotal reports and a pilot study have shown that anti-TNFα therapies may be beneficial for AD patients. However, as anti-TNFα therapeutic agents are biologics, their cost can be prohibitive. Apheresis may be prove to be a less expensive alternative, where media containing a TNFα antibody or binding partner can be used to remove a significant amount of TNFα with a relatively small amount of antibody or other binding partner.

In some embodiments, soluble TNF receptors are target molecules for apheresis of CSF, as soluble TNF receptors may be associated with Aβ metabolism and conversion to dementia in subjects with mild cognitive impairment. Any suitable TNF receptor, such as CD120a, CD120b or other TNF receptors may be a target molecule.

In various embodiments, one or more α- or γ-synuclein proteins are target molecules for CSF apheresis. Alpha- and γ-synuclein proteins have been found to be present in CSF and are increased in aged subjects with neurodegenerative and vascular changes. Alpha-synuclein is a structural component of Lewy body fibrils. Three point mutations have been identified in α-synuclein in some familial forms of Parkinson's disease (A53T, A30P, and E46K). In some embodiments, one or more of these mutated forms of α-synuclein may be target molecules for apheresis.

In some embodiments, Apolipoprotein E is a target molecule for apheresis of CSF. Apolipoprotein E may be a desirable target in any patient suffering from or at risk of AD or other dementia, and may be a particularly desirable target in patients carrying at least one allele of ApoE-ε4 or ApoE-ε3.

In some embodiments, BACE1 (also called β-secretase or memapsin-2) may be a target molecule for apheresis of CSF. BACE1, which cleaves at the β-site of amyloid precursor protein (APP), is thought to be involved in the pathogenesis of AD and other dementias. When APP is cleaved by BACE1 and γ-secretase results in the production of amyloid beta (Aβ), which is also a target molecule for CSF apheresis in some embodiments.

As used herein, “beta amyloid”, “amyloid beta”, “Abeta” and “Aβ” are used interchangeably. Aβ is peptide of about 39-43 amino acids that corresponds to a peptide formed in vivo upon cleavage of an amyloid beta A4 precursor protein (APP or ABPP) by beta-secretase (at the N-terminal portion of Aβ) and gamma secretase (at the C-terminal portion of Aβ). See, e.g., Strooper and Annaert (2000; J Cell Sci., 113, 1857-1870) and Evin and Weidemann (2002; Peptides, 23, 1285-1297). The most common isoforms of Aβ are Aβ40 and Aβ42, 40 and 42 amino acids, respectively. Aβ42 is less common, but is thought to be more fibrillogenic than Aβ40. Effective antibodies or binding partners may bind both Aβ40 and Aβ42, selectively bind Aβ42, bind all or some isoforms of Aβ, or the like.

Aβ is the main constituent of amyloid plaques in brains of Alzheimer's disease patients. Similar plaques can also be found in some Lewy body dementia patients and Down's Syndrome patients. Similar plaques or Aβ aggregates are found in the cerebral vasculature of cerebral amyloid angiopathy patients. More recent reports describe the accumulation of both soluble and intracellular Aβ ahead of the extracellular amyloid plaques forming (in all of the conditions above) in earlier disease states. In various embodiments, the systems, devices, or methods described herein may be employed to treat or prevent such diseases.

It will be understood that clearance of soluble forms of Aβ or fibrils or plaques containing Aβ are contemplated. Current models of the physical state of Aβ are evolving. Over about the last 20 years, researchers have defined the soluble toxic species of Aβ according to multiple synonyms. The antibodies described herein may target any of the species defined in Masters and Beyreuther's review (2006), Brain, November; 129(Pt 11):2823-39. Targets include soluble dimmers, tetramers, dodecomers that may ultimately form oligomers, oligomers, amorphous aggregates, Abeta derived diffusible ligands (ADDLS), β-balls, β-Amy balls, globular Aβ oligomer, paranuclei, preamyloid, protofibril, spherocylindrical miscelles, spherical particles, spherical prefibrillar aggregates, and toxic Aβ soluble species.

In some embodiments, a target molecule for apheresis of CSF is one or more of β-secretase (BACE)-cleaved soluble amyloid precursor proteins (sAPPβ), N-terminal fragments of APP, truncated APP or Aβ polypeptides, C-terminal truncated Aβ polypeptides, and the like.

In some embodiments, one or more of prostaglandin-d-synthase-transthyretin protein complex, isoprostane, toxica advanced glycation end-products (TAGE), and light chain, heavy chain or hyperphosphorylated heavy chain neurofilaments may be target molecules for apheresis of CSF. Each of these molecules may be associated with AD or other forms of dementia.

Binding Partner

Any suitable binding partner may be employed to remove a target molecule from CSF of a subject. A “binding partner” means any molecule which has selective binding affinity for the target molecule. Binding partners can include, without limitation, proteins, peptides, nucleic acids, amino acids, nucleosides, antibodies, antibody fragments, antibody ligands, aptamers, peptide nucleic acids, small organic molecules, lipids, hormones, drugs, enzymes, enzyme substrates, enzyme inhibitors, coenzymes, inorganic molecules, polysaccharides, and monosaccharides. As used herein, the term “selective binding affinity” means greater affinity for non-covalent physical association or binding to selected molecules relative to other molecules in a sample under appropriate conditions. Examples of selective binding affinity include the binding of polynucleotides to complementary or substantially complementary polynucleotides, antibodies to their cognate epitopes, and receptors to their cognate ligands under appropriate conditions (e.g., pH, temperature, solvent, ionic strength, electric field strength). Selective binding affinity is a relative term dependent upon the conditions under which binding is tested, but is intended to include at least a 2× greater affinity for amyloid beta than any non-target molecules present in a sample under appropriate conditions. If a test sample includes more than one type of target molecule (e.g., allelic variants from one locus), a binding partner can have selective binding affinity for one or more of the different target molecules relative to non-target molecules.

In many embodiments, a binding partner is an antibody, which can be readily produced or can be purchased from a commercial vendor such as Covance, Inc., Millipore, or AbD Serotec. Any antibody directed to a target molecule may be employed in accordance with the teachings presented herein. Exemplary antibodies include polyclonal, monoclonal, and humanized antibodies.

The term “antibody” is used in the broadest sense and specifically includes, for example, single monoclonal antibodies, antibody compositions with polyepitopic specificity, single chain antibodies, and fragments of antibodies (see below). The term “monoclonal antibody” as used herein refers to an antibody obtained from a population of substantially homogeneous antibodies, i.e., the individual antibodies forming the population are identical except for possible naturally-occurring mutations that may be present in minor amounts. An antibody may include an immunoglobulin constant domain from any immunoglobulin, such as IgG-1, IgG-2, IgG-3, or IgG-4 subtypes, IgA (including IgA-1 and IgA-2), IgE, IgD or IgM. In various embodiments, an antibody includes a combination of various immunoglobulin isotypes, either to a specific epitope of anti-amyloid or broader spectrum IgGs.

“Single-chain Fv” or “sFv” antibody fragments include the V_{H} and V_{L} domains of antibody, wherein these domains are present in a single polypeptide chain. Preferably, the Fv polypeptide further comprises a polypeptide linker between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The antibody may be directed towards any region of a target molecule. For example, for Aβ, antibodies may be directed to an epitope at the N-terminal region of Aβ, e.g., the epitope contains amino acids within 5 amino acids of the N-terminal amino acid. In some embodiments, the epitope lies within amino acids 3-8 of an Aβ peptide and corresponds to amino acids 1-17. In some embodiments, antibodies are directed at the mid-terminal region of Aβ, e.g., the epitope corresponds to amino acids 17-24 of human Aβ. In various embodiments, antibodies are directed to an epitope at the C-terminal region of Aβ, e.g., the epitope corresponds to amino acids 24-40/42/43 of human Aβ or contains amino acids within 5 amino acids of the C-terminal amino acid.

Any known or developed method for preparing antibodies may be used.

A. Polyclonal Antibodies

Polyclonal antibodies can be raised in a mammal, for example, by one or more injections of an immunizing agent and, if desired, an adjuvant. Typically, the immunizing agent or adjuvant will be injected in the mammal by multiple subcutaneous or intraperitoneal injections. The immunizing agent may include the target molecule or fragment or fusion protein thereof It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

B. Monoclonal Antibodies

Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the target molecule or fragment or fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell, e.g. as described in Goding, Monoclonal Antibodies: Principles and Practice, Academic Press, (1986) pp. 59-103. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Immortalized cell murine myeloma lines can be obtained, for example, from the Salk Institute Cell Distribution Center, San Diego, Calif. and the American Type Culture Collection, Manassas, Va. Human myeloma and mouse-human heteromyeloma cell lines also have been described for the production of human monoclonal antibodies See, e.g., Kozbor, J. Immunol., 133:3001 (1984); and Brodeur et al., Monoclonal Antibody Production Techniques and Applications, Marcel Dekker, Inc., New York, (1987) pp. 51-63.

The culture medium in which the hybridoma cells are cultured can then be assayed for the presence of monoclonal antibodies directed against the target molecule. For example, the binding specificity of monoclonal antibodies produced by the hybridoma cells can be determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods; e.g., as described in Goding, Monoclonal Antibodies: Principles and Practice, Academic Press, (1986) pp. 59-103. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (see, e.g., U.S. Pat. No. 4,816,567) or by covalently joining to the immunoglobulin coding sequence all or part of the coding sequence for a non-immunoglobulin polypeptide. Such a non-immunoglobulin polypeptide can be substituted for the constant domains of an antibody, or can be substituted for the variable domains of one antigen-combining site of an antibody to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art. For example, antibodies may be digested with papain digestion to form F(ab)′₂ fragments.

C. Human and Humanized Antibodies

Humanized forms of non-human (e.g., murine) antibodies may be chimeric immunoglobulins, immunoglobulin chains or fragments thereof (such as Fv, Fab, Fab′, F(ab′)₂ or other antigen-binding subsequences of antibodies) that contain minimal sequence derived from non-human immunoglobulin or that eliminate or reduce T-cell epitopes from the non-human antibodies. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also include residues which are found neither in the recipient antibody nor in the imported CDR or framework sequences. In general, the humanized antibody will include substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody may also include at least a portion of an immunoglobulin constant region (Fc), typically that of a human immunoglobulin. See, e.g., Jones et al., Nature, 321:522-525 (1986); Riechmann et al., Nature, 332:323-329 (1988); and Presta, Curr. Op. Struct. Biol., 2:593-596 (1992).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (see, e.g., Jones et al., Nature, 321:522-525 (1986); Riechmann et al., Nature, 332:323-327 (1988); and Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries. See, e.g., Hoogenboom and Winter, J. Mol. Biol., 227:381 (1991); and Marks et al., J. Mol. Biol., 222:581 (1991). Of course other techniques, such as those described by Cole et al. and Boemer et al,. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

The antibodies may also be affinity matured using known selection or mutagenesis methods. Affinity matured antibodies may have an affinity that is five time or more than the starting antibody (generally murine, humanized or human) from which the matured antibody is prepared.

Other methods for humanizing antibodies that may be employed include those described in, e.g., EO0629240, EP0983303, and PCT/GB06/000355, where methods for reducing or eliminating T cell epitopes are discussed.

In numerous embodiments, a humanized anti-Aβ antibody as described in U.S. Provisional Patent Application Ser. No. 60/990,401, entitled “Humanized Anti-Amyloid Beta Antibodies”, filed Nov. 27, 2007, and having attorney docket no. 30103.00 is employed according to the teachings presented herein.

Media

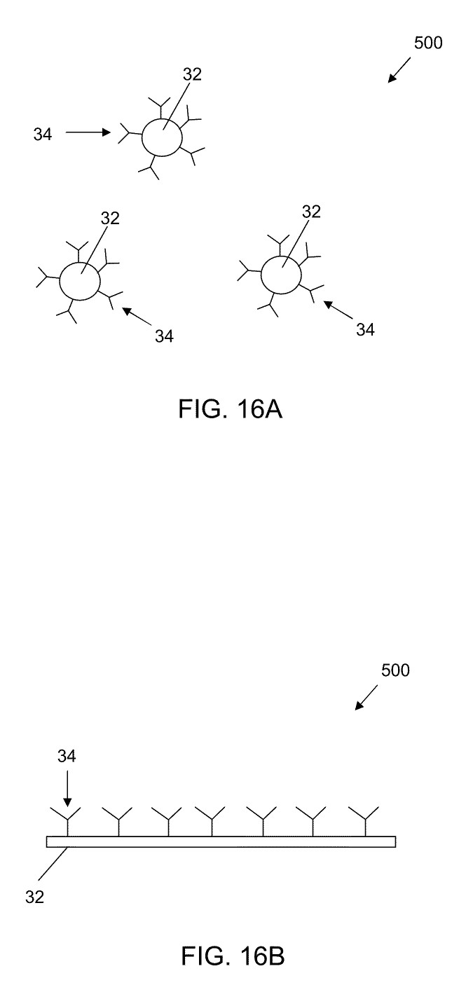

Referring now to FIGS. 16A-B, a medium 500 for removing a target molecule from CSF as described herein may include a solid support 32 and an antibody 34 or other suitable binding partner capable of selectively binding the target molecule. The solid support 32 may be porous or non-porous and may be of any convenient shape. For example, the solid support 32 may be in the form of beads (FIG. 16A), a membrane (FIG. 16B), hollow fiber, or the like. The solid support 32 may be made of any suitable material, such as glass, plastic polymer, polysaccharides, nylon, nitrocellulose, or TEFLON. Examples of suitable materials for beads include agarose, sepharose, dextran, polymethacrylate, polyacrylamide, silica and cellulose. In some embodiments the beads are paramagnetic, such as DynaBeads available from Baxter Immunotherapy Group, Santa Ana, Calif. Examples of suitable materials for membranes or fibers include cellulose, polysulfone, and polyamide. If the solid support 32 material is porous, the pores may be of any suitable size. In various embodiments, the pores are of a diameter of between about 300 to about 700 angstroms.

As shown in FIG. 16A-B, one or more antibody 34 or other suitable binding partner directed to the target molecule may be bound to a solid support 32 such as a bead or membrane. The antibodies may be bound to any surface of the support 32. For example, and while not shown in FIG. 16B, the antibodies 34 or binding partner may be bound to first and second opposing major surfaces of a membrane 32.

Antibodies or other suitable binding partners may be bound to the solid support via any suitable mechanism. As used herein, “binds”, “bind”, “bound”, “binding” or the like, in the context of an antibody 34 to a solid surface 32, refers to an association of the antibody 34 with the solid surface 32 that retains the antibody 34 in close proximity to the solid surface 32 when CSF flows through a reservoir containing media including the solid surface 32 with bound antibody 34.

The “binding” may be covalent or non-covalent. Examples of non-covalent binding include non-specific adsorption, binding based on electrostatic (e.g. ion, ion-pair interactions), hydrophobic interactions, hydrogen bonding interactions, surface hydration force and the like. Any suitable technique for non-covalently binding an antibody 34 or other suitable binding partner to a solid support 32 may be employed. For example, antibodies 34 may be attached to a solid support 32 by using protein A or G (bacterial cell wall proteins) which have high affinity to the constant (Fc) regions of antibodies. These proteins interface between the solid support 32 and the antibody 34. Protein A or G may be covalently attached to the solid support by using reductive amination, a cyanogen bromide technique, a gluteraldehyde method, or another suitable technique. Similarly, any suitable technique for covalently binding an antibody 34 or other suitable binding partner to a solid support 32 may be employed. Covalent immobilization of an antibody 34 or other suitable binding partners to a solid support 32 often involves activation of the antibody 34 or other suitable binding partners or the support 32. One example is the creation of aldehydes in the carbohydrate regions of an antibody 34 for its attachment to a support 32 that contains amines or hydrazide groups. Activation of the support 32 includes immobilization of antibodies 34 through their amine groups to supports 32 activated with N-hydoxysuccinimide or carbonyldiimidazole. Other methods used to link amine-containing antibodies 32 or other suitable binding partners to solid supports 34 include the cyanogen bromide method and reductive amination. Antibodies 34 or other suitable binding partners may also be attached to supports 32 using sulfhydryl-reactive methods which include haloacetyl, maliemide, and pyridyl disulfide methods. Antibodies 32 or other suitable binding partners may also be covalently linked to solid supports 34 using hydroxyl-reactive, carbonyl-reactive, or carboxyl-reactive methods.

In various embodiments, an Fc portion of an antibody 34 is bound to the solid support 32.

It will be understood that some antibody 34 may be eluted from the solid support 32 and may enter a CSF compartment of the subject with return of the CSF during apheresis. In such circumstances, it may be desirable to employ a humanized antibody when performing apheresis in a human to reduce the chance of developing an adverse immune reaction to the eluted antibody. In circumstances where little or no antibody 34 elutes from the solid support 32, it may be desirable to employ non-humanized antibodies as such antibodies are more readily obtainable in large quantities.

To maximize the capacity of the media 500 to remove the target molecule from CSF, the density of the antibody 34 or other suitable binding partner bound to the support 32 may be maximized. The density of the antibody 34 or other suitable binding partner bound to the support 32 can be readily controlled by varying the concentration of antibody 34 used to bind to the support 32.

When the media 500 containing the antibodies 34 or other suitable binding partners bound to the solid support 32 becomes saturated, fully or partially, with the target molecule removed from CSF, the media 500 may be replaced or regenerated. If the solid support 32 with bound antibody 34 or other suitable binding partners can flow through a syringe, such as with many beads, the media may readily be replaced. For example, a syringe needle or other suitable catheter may be inserted into a port 130 (see, e.g., FIGS. 5-6 and 14-15) to withdraw the medium containing the solid support 32 with bound antibody 34 or other suitable binding partners from a reservoir 30 and fresh medium may be introduced into the reservoir 30.

In various embodiments, solid surface is regenerated. As used herein, “regenerated”, in the context of media containing a solid support 32 with a bound antibody 34 or other suitable binding partners capable of binding a target molecule, means that the ability of the media to remove the target molecule is improved. Regeneration may include eluting the target molecule from the solid support with bound antibody. For example, an elution buffer may be added to the reservoir containing the media and later removed with eluted target molecule. Examples of solutions that may be used to elute target molecule from the antibody 32 include (i) low pH solutions (e.g., pH of about 1 to about 2.5) using, for example phosphate, citric, formic, or acetic acid, (ii) solutions having chaotropic agents, such as potassium or sodium thiocyanate at concentrations of about 1.5 M to about 3 M, sodium iodide at concentrations of about 2.5 M to about 3.0 M, or sodium chloride at concentrations of about 2M to about 4 M, and (iii) the like. It may be desirable to rinse the reservoir and media prior to resuming CSF flow through the reservoir. The reservoir may be rinsed with any physiologically acceptable solution, such as water, phosphate buffered saline, and the like.

The amount of target molecule removed from the media during regeneration of the media can be quantitatively or semi-quantitatively determined. Such information can be used to determine whether apheresis is effectively removing the target molecule, determine whether parameters should be altered (e.g., increase or decrease fluid flow through media, alter the concentration of the target molecule binding partner in the media, alter the specificity of the binding partner, etc.), or the like.

Methods of Treatment or Study

Apheresis of a target molecule from CSF of a subject may be employed to treat or study a variety of disease states. In various embodiments, apheresis as described herein is used to treat or study a disease associated with increased or aberrant soluble Aβ, amyloid fibrils or amyloid plaques. Examples of disease associated with increased or aberrant soluble Aβ, amyloid fibrils or amyloid plaques include Alzheimer's disease (AD), cerebral amyloid angiopathy (CAA), Lewy body dementia, and Down's Syndrome (DS).

In various embodiments a method includes identifying a subject suffering from or at risk of AD and removing Aβ or another target molecule from the patient's CSF via apheresis. Those at risk of AD include those of advancing age, family history of the disease, mutations in APP or related genes, having heart disease risk factors, having stress or high levels of anxiety. Identification of those suffering from or at risk of AD can be readily accomplished by a physician. Diagnosis may be based on mental, psychiatric and neuropsychological assessments, blood tests, brain imaging (PET, MRI, CT scan), urine tests, tests on the cerebrospinal fluid obtained through lumbar puncture, or the like.

In various embodiments a method includes identifying a subject suffering from or at risk of CAA and removing Aβ or another target molecule from the patient's CSF via apheresis. Symptoms of CAA include weakness or paralysis of the limbs, difficulty speaking, loss of sensation or balance, or even coma. If blood leaks out to the sensitive tissue around the brain, it can cause a sudden and severe headache. Other symptoms sometimes caused by irritation of the surrounding brain are seizures (convulsions) or short spells of temporary neurologic symptoms such as tingling or weakness in the limbs or face. CAA patients can be identified by, e.g., examination of an evacuated hematoma or brain biopsy specimen, the frequency of APOE ε2 or ε4 alleles, with clinical or radiographic (MRI and CT scans) grounds according the Boston Criteria (Knudsen et al., 2001, Neurology;56:537-539), or the like. Those at risk of CAA include those of advancing age, those having the APOE genotype, and those having other risk factors associated with AD.

In some embodiments a method includes identifying a subject suffering from or at risk of Down's Syndrome and removing Aβ or another target molecule from the patient's CSF via apheresis. A newborn with Down's Syndrome can be identified at birth by a physician's physical exam. The diagnosis may be confirmed through kariotyping. Multiple screening tests may be used to test or diagnosis a patient prior to birth (biomarkers, nuchal translucency, amniocentesis, etc.). A Down's Syndrome patient may be diagnosed with AD using diagnostic criteria relevant for AD.

In numerous embodiments a method includes identifying a subject suffering from or at risk of Lewy body dementia and removing Aβ or another target molecule from the patient's CSF via apheresis. Those suffering from or at risk of Lewy body dementia can be identified by mental, psychiatric or neuropyschological assessments, blood tests, brain imaging (PET, MRI, CT scan), urine tests, tests on the cerebrospinal fluid obtained through lumbar puncture, or the like. Those at risk of Lewy body dementia include those of advancing age.

In various embodiments, cerebral plaques may be cleared or prevented from forming by removing Aβ or another target molecule from the patient's CSF via apheresis. It will be understood that achieving any level of clearing of a plaque or plaques will constitute clearing of the plaque or plaques. It will be further understood that achieving any level of prevention of formation of a plaque or plaques will constitute preventing formation of the plaque or plaques. The methods may further include clearing or preventing parenchymal amyloid plaques or soluble forms of Aβ. The methods may further include improving cognitive aspects of the subject.

In some embodiments, cognitive abilities of a subject are improved by removing Aβ or another target molecule from the patient's CSF via apheresis.

In various embodiments, parenchymal amyloid plaques or soluble forms of Aβ are cleared in a subject by removing Aβ or another target molecule from the patient's CSF via apheresis.

The ability of a therapy described herein to treat a disease may be evaluated through medical examination, e.g. as discussed above, or by diagnostic or other tests. In various embodiments, a method as described in WO 2006/107814 (Bateman et al.) is performed. For example, a subject may be administered radiolabeled leucine. Samples, such as plasma or CSF, may then be obtained to quantify the labeled-to-unlabeled leucine in, for example, amyloid beta or other key disease related biomarkers, to determine the production and clearance rate of such proteins or polypeptides.

Clearing of, or formation of, amyloid beta can be evaluated in vivo by structural or functional neuro-imaging techniques. For example, diffusion tensor MRI (reviewed in Parente et al., 2008; Chua et al., 2008), PET imaging with the Aβ binding compound, Pittsburgh Compound B (PiB, Klunk et al., 2004; Fagan et al., 2006; Fagan et al 2007) or other SPECT based imaging of fibrillar Aβ structures and measurement of CSF levels of Aβ42 or tau may be employed. Distribution of vascular Aβ may be evaluated using differential interpretation of PET imaging of PiB (Johnson et al., 2007). Additionally, a cerebral microhemorrahage may be recognized by on gradient-echo or T-2 weighted MRI sequences (Viswanathan and Chabriat, 2006).

Similarly, detection of hemorrhages of the cerebral vasculature can be evaluated by imaging techniques, clinical evaluation, or the like. Spontaneous intracerebral hemorrhage (ICH) usually results in a focal neurologic deficit and is easily diagnosed on clinical and radiographic grounds (computed tomography (CT) scan, T-2 weighted MRI). Cerebral microhemorrhage results from underlying small vessel pathologies such as hypertensive vasculpathy or CAA. Cerebral microhemorrhages, best visualized by MRI, result from rupture of small blood vessels. The MRI diagnosis can be variable as described by Orgagozo et al., 2003 (Subacute meningoencephalitis in a subset of patients with AD after Aβ42 immunization-Elan Trial). For instance, patients showing signs and symptoms of aseptic meningoencephalitis MRIs showed only meningeal enhancement, whereas others had meningeal thickening, white matter lesions, with or without enhancing or edema, and a majority had posterior cerebral cortical or cerebellar lesions. Other potential diagnostics include changes in intracranial pressure, which may be detected by specific MRI techniques (Glick, et al., 2006, Alperin) or other standard techniques as described in Method of detecting brain microhemmorhage (U.S. Pat. No. 5,951,476).

Thus, embodiments of APHERESIS OF A TARGET MOLECULE FROM CEREBROSPINAL FLUID are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A system for removing a target molecule from cerebrospinal fluid of a subject, the system comprising:
a medium having a solid support to which an antibody directed to the target molecule is bound;
a reservoir for containing the medium;
a first catheter including a proximal end portion, an opening, and a lumen extending from the proximal end portion of the first catheter to the opening, wherein the first catheter is operably couplable to the reservoir such that fluid is capable of flowing from the lumen to the reservoir, and wherein the opening is configured to be placed in the cerebrospinal fluid of the subject; and
a second catheter including a proximal end portion, a delivery region and a lumen extending from the proximal end portion of the second catheter to the delivery region, wherein the second catheter is operably couplable to the reservoir such that fluid is capable of flowing from the reservoir to the lumen, and wherein the delivery region is configured to be placed in the cerebrospinal fluid of the subject.

2. The system of claim 1, wherein the target molecule is amyloid beta.

3. The system of claim 1, further comprising:
a pump operably couplable to the first catheter, second catheter, and the reservoir, wherein the pump is configured to cause cerebrospinal fluid to flow from the opening of the first catheter through the reservoir and to the delivery region of the second catheter.

4. The system of claim 1, further comprising a filter positioned and configured to allow cerebrospinal fluid to flow from the reservoir to the lumen of the second catheter and to prevent flow of the medium from the reservoir to the lumen of the second catheter.

5. The system of claim 1, wherein the solid support comprises beads.

6. The system of claim 5, wherein the beads comprise pores having a diametric dimension of between about 300 to about 700 angstroms.

7. The system of claim 1, wherein the medium comprises a membrane and wherein the antibodies are bound to the membrane.

8. The system of claim 1, wherein the medium comprises a fiber and wherein the antibodies are bound to the fiber.

9. The system of claim 1, further comprising a housing, wherein the reservoir is disposed within the housing.

10. The system of claim 9, wherein the housing is hermetically sealed and is configured to be implanted within the subject.

11. The system of claim 10, further comprising a port providing access to the reservoir from outside the housing.

12. The system of claim 1, wherein a single multi-lumen catheter comprises the first and second catheters.

13. A method for removing a target molecule from cerebrospinal fluid of a subject, the method comprising:
withdrawing fluid from a first cerebrospinal fluid compartment of the subject;
passing the withdrawn fluid through a reservoir of a medical device to remove the target molecule from the withdrawn fluid, wherein the reservoir contains a medium comprising a solid support to which an antibody directed to the target molecule is bound and wherein the media is capable of removing the target molecule from the cerebrospinal fluid;
returning the withdrawn fluid with removed target molecule to a second cerebrospinal fluid compartment of the subject,
wherein the first and second cerebrospinal fluid compartments are the same or different.

14. The method of claim 13, wherein the target molecule is amyloid beta.

15. The method of claim 13, further comprising filtering the media from the withdrawn fluid with removed target molecule prior to returning the withdrawn fluid with removed target molecule to the second cerebrospinal fluid compartment of the subject.

16. The method of claim 13, further comprising regenerating the medium to increase the capacity of the medium to remove the target molecule from the withdrawn fluid.

17. The method of claim 13 further comprising removing the medium from the reservoir and placing fresh medium in the reservoir.

18. An implantable medical device comprising:
a housing;
a reservoir disposed in the housing;
a medium disposed in the reservoir and comprising a solid surface to which antibodies directed to a target molecule are bound;
a fluid outlet operably coupled to the reservoir;
a fluid inlet operably coupled to the reservoir; and
a pump disposed in the housing, operably coupled to the fluid inlet, the fluid outlet, and the reservoir, and configured to cause fluid to flow from the inlet through the reservoir and to the outlet.

19. The implantable device of claim 18, wherein the target molecule is amyloid beta.

20. The implantable device of claim 18, further comprising a port providing access to the reservoir from outside the housing.

21. A system comprising:
the implantable medical device of claim 18;
a first catheter including a proximal end portion, an opening, and a lumen extending from the proximal end portion to the opening, wherein the first catheter is operably couplable to the fluid inlet such that fluid is capable of flowing from the lumen to the inlet, and wherein the opening is configured to be placed in the cerebrospinal fluid of the subject; and
a second catheter including a proximal end portion, a delivery region and a lumen extending from the proximal end portion to the delivery region, wherein the second catheter is operably couplable to the fluid outlet such that fluid is capable of flowing from the outlet to the lumen, and wherein the delivery region is configured to be placed in the cerebrospinal fluid of the subject.

22. The system of claim 21, wherein a single multi-lumen catheter comprises the first and second catheters.
